(19) United States
(12) Patent Application Publication
(10) Pub. No.: US 2010/0075419 A1
(43) Pub. Date: Mar. 25, 2010

(54) BIOMATERIAL, METHOD OF CONSTRUCTING THE SAME AND USE THEREOF

(76) Inventors: Masahiko Inagaki, Aichi (JP); Akira Watazu, Aichi (JP)

Correspondence Address: OBLON, SPIVAK, MCCLELLAND MAIER & NEUSTADT, L.L.P., 1940 DUKE STREET, ALEXANDRIA, VA, 22314, US

(21) Appl. No.: 12/442,860

(22) PCT Filed: Sep. 25, 2007
(86) PCT No.: PCT/JP07/68585

§ 371 Date: Aug. 18, 2009

(30) Foreign Application Priority Data
Sep. 26, 2006 (JP) 2006-259915
Sep. 21, 2007 (JP) 2007-246247

(51) Int. Cl. B32B 3/26; B29C 41/42; C12N 5/00; B29C 33/42; A61F 2/02; A61F 2/28

(52) U.S. Cl. 435/402; 428/304.4; 428/213; 428/315.5; 264/334; 249/187.1; 623/11.11; 623/23.5

(57) ABSTRACT

The present provides a biomaterial composed in part of a porous material having an internal structure that has been completely controlled so as to optimize living tissue infiltration or cell introduction, a method of manufacturing, and uses thereof, including bio-implant materials for artificial bones, artificial joints and artificial tooth roots, and cell culture supports; the biomaterial undergoes increased infiltration by living tissues and the like owing to the formation of a porous region in at least a portion of the material, wherein the porous region is a porous body having therein a group of oriented pores that has an orientation and is made up of pores whose size, shape and direction have been controlled to optimize living tissue infiltration or cell introduction, and also having formed therein connecting pores that link together the primary pores and enable the passage of bodily fluids and gas bubbles, and formed with a spatial configuration in which the oriented pores are not directly connected to other oriented pores and the connecting pores which link together the oriented pores are not directly connected to other connecting pores.

### TECHNICAL FIELD

The present invention relates to a porous biomaterial and a method of manufacture thereof. More specifically, the invention relates to a bio-implant material, e.g., artificial bone, artificial joint, artificial tooth root, or cell culture support in which have been formed, at the interior of a porous body, connecting pores which are controlled for orientation, size and shape thereof, and to a method of manufacture thereof; and this bio-implant material or cell culture support is characterized by having formed, at the interior of a porous body, a group of oriented pores controlled for pore size, shape and direction thereof and connecting pores which link together the oriented pores. The present invention provides, in the technical field of biomaterials, a novel type of biomaterial, e.g., bio-implant material, cell culture support, dialysis component, circulation device component, or filter, which is a porous biomaterial having formed, at the interior thereof, pores control for orientation, size, shape and direction thereof, and which has strength, mechanical characteristics and anisotropy of the propagation of vibrations and the like, and also which enables infiltration by living tissue or the introduction of cells.

### BACKGROUND ART

In living tissues, the formation of a variety of ordered structures is seen at various sites, from the macroscopic level down to micrometer and even nanometer sizes. These ordered structures manifest advanced functions which include protecting the vital organs of the body, supporting the limbs, and imparting to the skeleton sufficient strength for movement. There is an expectation that, were it possible to reconstruct these highly ordered structures in living tissue, advanced biomaterials which possess functions essential to the tissue at the site of implantation and are closer to the advanced functions of the living body could be created.

In living bone, for example, as observed near the femoral head, optimal stress dispersion is achieved through the orientation of the trabeculae within the spongy substance. In a porous body as well, anisotropy arises in the mechanical characteristics (strength, modulus) of the body through the control of the geometric shape and distribution (i.e., shape and orientation) of the pores. Hence, it is thought to be possible to achieve a new type of implantable material which is capable of dispersing stress in the same way as living bone at the site of implantation.

The bones and skeleton in a human body have various functions in order to work in harmony with the surrounding muscles, internal organs, nervous tissue, etc. at various places within the body. One function of the bones and skeleton is to support loads owing to the weight of the body and movement, and to protect the internal organs. The skeletal structure is ideal for carrying out this function. Each bone has a shape and internal structure suitable for dispersing stress at that site. Because artificial bones used at sites which are subjected to loads are required to have a high strength, metal bodies or compact ceramic bodies are employed for this purpose. However, because such artificial bones have mechanical characteristics (e.g., Young's modulus) which differ considerably from those of living bone, a good mechanical match with bone at the site of implantation and with surrounding bone connected thereto as part of the skeleton has not been achieved.

This gives rise to problems such as the destruction of cartilage and the loss of bone mass in surrounding bones and joints due to stress concentration. By making the material porous, it can be mechanically matched with the living bone, although there is a need in such a case to control the interior structure made up of pores and walls. Also, in artificial bone, when the pores at the interior are isolated, this impedes the passage of bodily fluids, etc., restricting the supply of nutrients and oxygen. As a result, the infiltration by bone and other tissue is inadequate, hindering tissue regeneration. Also, when gas bubbles that have nowhere to go remain within the pores, they can hamper cellular, tissue and vascular infiltration. Therefore, to prevent the isolation of pores at the interior, it would be desirable to have the ability to controllably form a connected structure.

Use is currently being made of bio-implants which, by making a substrate composed of metal, ceramic or polymer porous, have been designed so that living tissue such as bone tissue will infiltrate into the pores. The dimensions, shape, orientation and other geometric properties of the pores are known to exert an influence on the living tissue that forms there. For example, it has been reported that, in honeycomb-shaped hydroxyapatite, the difference between whether direct bone formation takes place within the pores or cartilaginous bone formation occurs depends on the pore diameter (see Non-Patent Document 1).

It has also been reported that when 100 perforations are formed with a laser at fixed intervals in a collagen film, Haversian bone such as is seen in the cortical bone of the femoral diaphysis forms (see Non-Patent Document 2). These reports clearly show that the geometric structure of artificial objects of a type that is infiltrated by living tissue and serves as scaffolding contributes significantly to the reconstruction of the highly ordered structure of living tissue.

However, in these reports, the only artificial objects serving as scaffolding that are mentioned are very small honeycomb shaped bodies or thin sheets in which perforations have been formed. For use as an actual implant material such as artificial bone, a bulk porous body which is effective on all the constituent elements of the body, including hard tissue, soft tissue and bodily fluids, and in which the size, shape and direction of the pore spaces that manifest the necessary functions at the site of implantation have been controlled, is required. For example, the presence of a large number of pores is desirable for the ease of flow of bodily fluids and the like; even a small pore size is acceptable for the passage of bodily fluids, etc.

However, pores having a size of about 100 μm are necessary for bone tissue and vascular infiltration. On the other hand, from the standpoint of the strength of the porous body, it is necessary for the walls which form the porous body to have a configuration which affords the required strength. Therefore, to achieve a porous artificial bone of a sort which is capable of inducing the ordered structure (oriented structure) of tissue such is seen within the living body, which is mechanically matched with the surrounding bone and which has the necessary strength, a technique is needed that can construct such artificial bone while controlling in detail the orientation of the pores and the internal structure composed of pore connections and walls.

Prior-art relating to the formation of bulk porous bodies includes, as methods of forming three-dimensional communicating pores, processes which use powders of differing particle sizes, dissolving some or all of the fine particles and depositing the coarse particles on a substrate (see Patent Documents 1 and 2), and processes which add a blowing agent to a slurry or the like and form a foam. Because the formation of open pores is probabilistic, it is impossible to directly control the orientation, size and shape of the pores. Also, probabilistically, there is a possibility that closed pores will form. The existence of closed pores presents a danger of gas bubbles being released within the body should breakage of the biomaterial occur. Moreover, the inability of bodily fluids, cell culture fluids, cells and tissue to infiltrate closed pores limits the utility of such porous bodies in tissue repair, tissue engineering and regenerative medicine. Hence, such processes are unsuitable as methods for manufacturing biomaterials.

In addition, methods involving the lamination of a mesh or the like (see Patent Documents 3 to 6) have been proposed. A honeycomb-like porous body that communicates in one direction only (see Patent Document 4) and porous bodies in which the pore shapes are isotropic and have no orientation (see Patent Documents 3, 4 and 6) can be formed. However, in a honeycomb-like arrangement of communicating pores, each pore is independent, which is undesirable for bone tissue infiltration. Moreover, a porous body without orientation is poorly suited for controlling the morphology of the living tissue that is to be formed there.

A process of forming macroscopically oriented pores by using an aqueous ceramic slurry or a slurry to which has been added an aqueous solution containing an element that acts as a sintering aid, causing ice to grow unidirectionally during freezing, then vacuum drying so as to form pores as vestigial traces of ice sublimation, and sintering the resulting porous shaped body (see Patent Document 7) has also been disclosed. However, given that the size of the ice which forms during freezing and which has grown so as to be macroscopically oriented determines the size and shape of the pores, while some control of the size of the pores by the ice growth conditions is possible, a porous body in which the shape and size have been completely controlled cannot be formed.

Methods of forming a porous body in which throughholes have been formed, by arraying a plurality of columnar cells without overlap within a plane, stacking thereon other columnar cells having a different direction of orientation, packing calcium phosphate cement into interstices between the columnar cells and curing the cement, then removing the columnar cells (Patent Documents 8 and 9) have also been disclosed. However, because the columnar cells are stacked so as to have different directions of orientation, one problem is that bi-directional throughholes are inevitably formed. Another problem is that, in the process of forming a porous body in which throughholes have been formed, the direction having a relatively good strength to loading becomes fixed in a direction perpendicular to the direction of the throughholes. Yet another problem is that, owing to constraints having to do with the production steps, this process can only be applied to low-temperature curing calcium phosphate shaped bodies.

Also, the fact that the oriented throughholes are in mutual contact and directly connected with each other does not lend itself well to control of the spatial configuration of the oriented holes, resulting in the additional problem that the shape of the holes in the areas of contact cannot be controlled. A process of forming a porous body having oriented pores formed therein by bringing together throughhole-bearing ceramic spheres so that the throughholes are oriented in one direction (see Reference Document 10) has also been described. However, in such a method of forming a porous body, all that can be obtained is a structure in which the oriented pores are scattered within a network of pores formed in the gaps between the very small spherical units; also, the spatial size of the main pores formed is limited by the size of the units.

Also, because the gaps between the units invariably become connecting pores, another problem is the formation of unnecessary connecting pores. This type of approach is thus inconvenient in the design of, for example, strength, mechanical characteristics, control of the propagation of vibrations, and optical properties. In addition, when spherical units are brought together, because the units join together at the points where spheres come into contact with other spheres, forming a porous body having a high strength is difficult.

To form a porous artificial bone which contributes to the formation of ordered living tissue and is compatible with the mechanical characteristics at the site of implantation, there is a desire for a structure which controls the direction of the primary pores so as to be oriented in any desired direction and moreover has formed therein connecting pores that allow the passage of bodily fluids and gas bubbles and link together the primary oriented pores. However, a biomaterial which, in order to be advantageous for infiltration by living tissue and for the introduction of cells, is formed so as to be three-dimensionally porous with a structure having a controlled spatial configuration composed of a group of oriented pores having pores spaces of controlled size, shape and direction and connecting pores which link together the oriented pores has yet to be achieved.

### Patent Document 1: Japanese Patent No. 2710849

Patent Document 2: Japanese Patent Application Laid-open No. H5-056990

### Patent Document 3: Japanese Patent No. 3243679

### Patent Document 4: Japanese Patent No. 3261030

Patent Document 5: Japanese Patent Application Laid-open No. H7-171172
Patent Document 6: Japanese Patent Application Laid-open No. H8-173463

### Patent Document 7: Japanese Patent Application Laid-open No. 2001-192280

### Patent Document 8: Japanese Patent Application Laid-open No. 2005-46530

### Patent Document 9: Japanese Patent Application Laid-open No. 2004-261456

### Patent Document 10: Japanese Patent Application Laid-open No. 2003-335574

Non-Patent Document 1: Kuboki et al., J. Bone Joint. Surg. 83-A, S1-105-115 (2001)

### Non-Patent Document 2: Kikuchi et al., J. Hard Tissue Biol. 9, 79-89 (2000)

In light of this situation and the above-described prior-art, the inventors have conducted extensive and repeated studies aimed at the development of a porous bio-implant material which is characterized by being a porous body having formed at the interior thereof a group of oriented pores in which the size, shape and direction of the individual pore spaces have been controlled and also connecting pores which allow the passage of bodily fluids and gas bubbles and link together the oriented pores, and which is also characterized by having anisotropy of strength, of mechanical properties, and of the propagation of vibrations, etc., enabling infiltration by living tissue and the introduction of cells.

As a result, the inventors have discovered that, by at least controlling the spatial configuration of the group of oriented pores and the connecting pores which link together the oriented pores in such a way that the oriented pores are spatially configured so as not to be directly connected to other oriented pores and the connecting pores which link together the oriented pores are spatially configured so as not to be directly connected to other connecting pores, it is possible to form a porous bio-implant material having present at the interior of a porous body a group of oriented pores in which the size, shape and direction of the individual pore spaces have been controlled and also having connecting pores, which material has anisotropy of strength, mechanical properties and the propagation of vibrations, etc., and allows infiltration by living tissue and the introduction of cells. This discovery ultimately led to the present invention.

### DISCLOSURE OF THE INVENTION

It is therefore an object of the present invention to provide a porous bio-implant material having present therein both a group of oriented pores having an orientation and also connecting pores, the size, shape and direction of each pore space at the interior of the porous body being controlled, which material has anisotropy of strength, mechanical properties and the propagation of vibrations, etc., and enables infiltration by living tissue and the introduction of cells. Another object of the invention is to provide a method of manufacturing such a material.

The present invention for resolving the above problems is technically constituted as follows.
(1) A porous biomaterial with controlled orientation, which biomaterial is characterized (1) by having a group of oriented pores, at least 50% of which in a long axis direction is oriented in the same direction, (2) by having connecting pores that are formed so as to link together the oriented pores, and enabling the passage of bodily fluids and gas bubbles, and (3) by spatial configuration in which the oriented pores are not directly connected to other oriented pores and the connecting pores which link together the oriented pores are not directly connected to other connecting pores.
(2) The biomaterial of (1) above, which is made of metal, polymer, ceramic or a composite of any two or more thereof.
(3) The biomaterial of (1) above, which is formed of stacked sheets.
(4) The biomaterial of (3) above, wherein each sheet to be stacked has a thickness of from 10 μm to 2 mm, or up to one-half the entire thickness of the biomaterial.
(5) The biomaterial of (3) above, wherein the sheets to be stacked have a pore size with a minimum width, in a direction perpendicular to the surface of the sheets, in a range of from 0.1 μm to 1 mm.
(6) The biomaterial of (3) above, wherein the sheets to be stacked have a pore size with a maximum width, in a direction perpendicular to the surface of the sheets, in a range of from 10 μm to 10 mm.
(7) The biomaterial of (3) above, wherein the sheets to be stacked have a pore frequency of from 1 to 250,000 per square centimeter.
(8) The biomaterial of (3) above, wherein the sheets to be stacked are made of metal, polymer, ceramic or a composite of any two or more thereof.
(9) The porous biomaterial of any of (1) to (8) above, wherein at least a portion of walls of the oriented pores and/or connecting pores contains, or is covered with, at least one selected from among calcium phosphate, titanium oxide, alkali titanates, polymers, silane coupling agents, compounds formed by hydrolyzing a metal alkoxide, mesoporous materials, drugs, and compounds containing one or more element from among calcium, magnesium, sodium, potassium, lithium, zinc, tin, tantalum, zirconium, silicon, niobium, aluminum, iron, phosphorus and carbon.
(10) The biomaterial of (9) above, wherein at least a portion of the walls of the oriented pores and/or connecting pores has been rendered porous by anodization.
(11) The biomaterial of (9) above, wherein at least a portion at the interior of the oriented pores and the connecting pores which link together the oriented pores holds at least one type of filler composed of one or more selected from metal, ceramic, polymer or a composite thereof.
(12) The biomaterial of (9) above, wherein at least a portion at the interior of the oriented pores and the connecting pores which link together the oriented pores holds at least one type of particle composed of one or more selected from metal, ceramic, polymer or a composite thereof.
(13) A process for manufacturing the porous biomaterial of any of (1) to (8) above, comprising: using as a mold a shaped body, which has a structure obtained by stacking and joining together sheets containing pores of at least two types of shape, array pattern and frequency, the pores being of differing width-to-length ratios, while controlling hole positions in the sheets, and which has, at the interior of a porous body, a group of oriented pores of individually controlled size, shape and direction, and is formed therein with connecting pores linking together the oriented pores, and is formed with a spatial configuration in which the oriented pores are not directly connected to other oriented pores and the connecting pores which link together the oriented pores are not directly connected to other connecting pores; filling the pores with a slurry of metal, ceramic, polymer or a composite thereof; then removing the shaped body serving as the mold by sintering or by dissolution with a solvent so as to produce a biomaterial which has, at the interior of a porous body, a group of oriented pores of individually controlled size, shape and direction, is formed therein with connecting pores linking together the oriented pores, and is formed with a spatial configuration in which the oriented pores are not directly connected to other oriented pores and the connecting pores which link together the oriented pores are not directly connected to other connecting pores.
(14) A process for manufacturing a porous biomaterial, comprising: casting a metal or a ceramic particle-containing metal by using, as a lost wax mold, a shaped body, which has a structure obtained by stacking and joining together sheets containing pores of at least two types of shape, array pattern and frequency, the pores being of differing width-to-length ratios, while controlling pore positions in the sheets, and which has, at the interior of a porous body, a group of oriented pores of individually controlled size, shape and direction, is formed therein with connecting pores linking together the oriented pores, and is formed with a spatial configuration in which the oriented pores are not directly connected to other oriented pores and the connecting pores which link together the oriented pores are not directly connected to other connecting pores, so as to produce a biomaterial which has, at the interior of a porous body, a group of oriented pores of individually controlled size, shape and direction, has formed therein connecting pores linking together the oriented pores, and is formed with a spatial configuration in which the oriented pores are not directly connected to other oriented pores and the connecting pores which link together the oriented pores are not directly connected to other connecting pores.
(15) A bio-implant, which at least partially comprises the biomaterial of any one of (1) to (12) above.
(16) The bio-implant of (15) above, which has a three-dimensional trabecular structure derived from a mechanical model of a bone.
(17) A cell medium support, which at least partially comprises the biomaterial of any one of (1) to (12) above.
(18) A mold for the porous biomaterial of (1) to (12) above, the mold comprising a shaped body which has a structure obtained by stacking and joining together sheets containing pores of at least two types of shape, array pattern and frequency, the pores being of differing width-to-length ratios, while controlling pore positions in the sheets, and which has, at the interior of a porous body, a group of oriented pores of individually controlled size, shape and direction, is formed therein with connecting pores linking together the oriented pores, and is formed with a spatial configuration in which the oriented pores are not directly connected to other oriented pores and the connecting pores which link together the oriented pores are not directly connected to other connecting pores.
(19) The biomaterial of (1) above, which is formed therein with holes other than the oriented pores and the connecting pores formed so as to link together the oriented pores.
(20) The biomaterial of (1) above, which is of a size where the minimum length of the oriented pores in any cross-section is from 1 to 1,000 μm.
(21) The biomaterial of (1) above, wherein the porous biomaterial is a shock absorbing material which has been controlled to a size where the minimum length of the oriented pores in any cross-section is from 1 to 30 mm.
(22) The biomaterial of (1) above, which is made of titanium or a titanium alloy.
(23) The biomaterial of (1) above, which is made of calcium phosphate.

Next, the present invention is described in greater detail.

The present invention relates to a biomaterial which, by having formed in at least some portion thereof a porous region of controlled orientation, increases infiltration by living tissue and the like, and is thus a material that increases the ability for essential bodily functions to appear at the site of implantation. The inventive biomaterial is characterized (1) in that the porous region has a group of oriented pores of controlled size and shape, enabling infiltration by living tissue and the introduction of cells, (2) in that connecting pores which link together the oriented pores and allow the passage of bodily fluids and gas bubbles have been formed therein, and (3) by being formed in a manner where the oriented pores are spatially configured so as not to be directly connected to other oriented pores and the connecting pores which link together the oriented pores are spatially configured so as not to be directly connected to other connecting pores.

The porous biomaterial of the invention does not have formed therein linkages between connecting pores of the type formed by gaps between beads. Nor does it have formed therein connections between oriented pores of a type obtained by a method which involves arraying a plurality of columnar cells without overlap within a plane, stacking thereon columnar cells arrayed in a different direction, filling calcium phosphate cement into interstices between the columnar cells and setting the cement. Holes which are formed by a sintering process or the like may be formed in the porous body of the invention.

Also, the present invention is characterized in that, in the above-mentioned porous biomaterial, at least some portion of the walls of the oriented pores and/or connecting pores contains, or is covered with, at least one substance selected from among calcium phosphate, titanium oxide, alkali titanates, polymers, silane coupling agents, compounds formed by hydrolyzing a metal alkoxide, mesoporous materials, drugs, and compounds containing one or more element from among calcium, magnesium, sodium, potassium, lithium, zinc, tin, tantalum, zirconium, silicon, niobium, aluminum, iron, phosphorus and carbon.

In one aspect of the present invention, a shaped body with a structure obtained by stacking and joining together sheets containing holes of at least two types of shape, array pattern and frequency, the holes being of differing width-to-length ratios, while controlling the hole positions in the sheets, which shaped body has, at the interior of a porous body, a group of oriented pores of individually controlled size, shape and direction, has formed therein connecting pores which link together the oriented pores, and is formed in such a way that the oriented pores are spatially configured so as not to be directly connected to other oriented pores and the connecting pores which link together the oriented pores are spatially configured so as not to be directly connected to other connecting pores, is used as a mold.

This aspect of the invention is characterized by using such a shaped body as a mold and filling the pores with a slurry of metal, ceramic, polymer or a composite thereof, then removing the shaped body serving as the mold by sintering or by dissolution with a solvent so as to manufacture a biomaterial which has, at the interior of a porous body, a group of oriented pores of individually controlled size, shape and direction, has formed therein connecting pores which link together the oriented pores, and is formed in such a way that the oriented pores are spatially configured so as not to be directly connected to other oriented pores and the connecting pores which link together the oriented pores are spatially configured so as not to be directly connected to other connecting pores.

In another aspect of the invention, a shaped body with a structure obtained by stacking and joining together sheets containing holes of at least two types of shape, array pattern and frequency, the holes having differing width-to-length ratios, while controlling the hole positions in the sheets, which shaped body is characterized by having, at the interior of a porous body, a group of oriented pores of individually controlled size, shape and direction, has formed therein connecting pores which link together the oriented pores, and is formed in such a way that the oriented pores are spatially configured so as not to be directly connected to other oriented pores and the connecting pores which link together the oriented pores are spatially configured so as not to be directly connected to other connecting pores, is used as a lost wax mold.

This aspect of the invention is characterized by using such a shaped body as a lost wax mold to cast a metal or a ceramic particle-containing metal so as to manufacture a biomaterial which has, at the interior of a porous body, a group of oriented pores of individually controlled size, shape and direction, has formed therein connecting pores which link together the oriented pores, and is formed in such a way that the oriented pores are spatially configured so as not to be directly connected to other oriented pores and the connecting pores which link together the oriented pores are spatially configured so as not to be directly connected to other connecting pores.

In other aspects, the invention is characterized by a bio-implant which is at least partially composed of the above-described biomaterial, and by a cell medium support which is at least partially composed of the above-described biomaterial. In a still further aspect, the invention is characterized by a mold for the above-described porous biomaterial, which mold is a shaped body with a structure obtained by stacking and joining together sheets containing holes of at least two types of shape, array pattern and frequency, the holes being of differing width-to-length ratios, while controlling the hole positions in the sheets, which shaped body has, at the interior of a porous body, a group of oriented pores of individually controlled size, shape and direction, has formed therein connecting pores which link together the oriented pores, and is formed in such a way that the oriented pores are spatially configured so as not to be directly connected to other oriented pores and the connecting pores which link together the oriented pores are spatially configured so as not to be directly connected to other connecting pores.

The present invention relates to a porous biomaterial, a method of manufacturing the same, and uses thereof. More particularly, the invention relates to a biomaterial which, by having formed in at least some portion thereof a porous region of controlled orientation, increases infiltration by living tissue and the like, and is thus a material that increases the ability for essential bodily functions to appear at the site of implantation. The inventive biomaterial is characterized (1) in that the porous region has a group of oriented pores of controlled size and shape, enabling infiltration by living tissue and the introduction of cells, (2) in that connecting pores which link together the primary pores and allow the passage of bodily fluids and gas bubbles have been formed therein, and (3) by being formed in a manner where the oriented pores are spatially configured so as not to be directly connected to other oriented pores and the connecting pores which link together the oriented pores are spatially configured so as not to be directly connected to other connecting pores. The invention also relates to a method of manufacturing such biomaterials, and uses for the biomaterials.

In the present invention, the porous biomaterial may be utilized in, for example, bio-implant materials, cell culture supports, dialysis components, circulation device components and filters, but is not limited to these uses. In this invention, the term “bio-implant material” refers to a shaped body which forms a porous layer on the outside or inside of part or all of the surface of a bio-implant material substrate, and is typically used in vivo as, for example, artificial bone, artificial joint or artificial tooth root.

The bio-implant material is not subject to any particular limitation with respect to shape, manner or use, etc., provided it has the properties and safety required for use in vivo. The bio-implant material of the invention may have any shape, such as a block-like, columnar, plate-like, or amorphous bulk-like shape. The manner of use for the inventive bio-implant material includes such product configurations as artificial joint stems, artificial knee joints, artificial vertebral bodies, artificial intervertebral disks, bone filling materials, bone plates, bone screws and artificial tooth roots.

In the present invention, “cell culture support” refers to a shaped body for culturing cells or tissue in cell engineering, tissue engineering or regenerative medicine. So long as it has the properties required for use in cell culturing, the shape and manner of use are not subject to any particular limitation. For example, use may be made of any shape, such as a plate-like, sheet-like, block-like, columnar, amorphous bulk-like, or cup-like shape. The manner of use includes product configurations such as Petri dishes for cell cultivation and sheets for cell cultivation.

Preferred examples of the metal used in the invention include pure titanium, titanium alloys, stainless steel, cobalt (Co) and cobalt alloys, tantalum (Ta), niobium (Nb) and their alloys, and gold (Au), silver (Ag), copper (Cu) and platinum (Pt). Preferred examples of the ceramic used in the invention include calcium phosphate-based ceramics such as hydroxyapatite and calcium triphosphate, alumina-based ceramics, zirconia-based ceramics, silicon-based ceramics, titania-based ceramics, glasses for biomaterials which contain at least calcium and phosphorus, and crystallized glass for biomaterials.

Preferred examples of the polymer used in the invention include polyolefin (co)polymers; polystyrene polymers; polyvinyl chloride and polyvinylidene chloride-based polymers; polyvinyl alcohol, polyvinyl alcohol ester and polyvinyl acetal-based polymers; polymers of unsaturated compounds in which nitrogen atoms on substituents are directly bonded to the aliphatic chain; polymers of unsaturated compounds in which carbonyl groups or nitrile groups are directly bonded to an aliphatic chain, such as poly(meth)acrylic acid (ester) polymers, poly(meth)acrylonitrile polymers and poly(meth)acrylamide polymers; polycyanoacrylate polymers; polydiene polymers; fluorocarbon resins; and polyester polymers.

Further examples of the polymer used in the present invention include hydroxycarboxylic acid-based polymers such as polylactic acid, polyether or polyoxide-based polymers, polyether/polyester polymers, polycarbonate polymers, polyurethane(urea) polymers, segmented polyurethane(urea) polymers, polyamide or polyimide-based polymers, polyamino acid-based polymers, polyacetal polymers, silicon-containing polymers, and sulfur-containing polymers.

Still further examples of the polymer include cellulose and cellulose derivatives, starch and starch derivatives, agarose and agarose derivatives, polysaccharides such as agar, alginic acid and gums, heparin and heparin derivatives, chondroitin and chondroitin derivatives, mucopolysaccharides such as hyaluronic acid, chitin and chitosan, collagen and collagen derivatives such as atelopeptide collagen and reconstituted fiber collagen, gelatins, keratin, and copolymers, block copolymers, graft polymers, crosslinked forms, or composites thereof, of any two or more of the above polymers.

The sheets for stacking which are used in the invention are preferably composed of one or more of the following: metals, ceramics, polymers, carbonaceous materials, and composites thereof. These composites are materials composed of two or more types of mutually differing substances which are strongly bonded and united by physical, chemical or mechanical mixing and joining. Illustrative examples include composite materials obtained by kneading together components made of different substances, composite materials obtained by precipitation from a precursor solution or the like, materials obtained by joining together components made of different substances, and materials obtained by depositing a thin layer of a substance on a substrate to form an integral body.

Preferred, non-limiting, examples of the drug used in the present invention include anti-inflammatory agents, fibronectins, albumins and laminins, clotting and anti-clotting factors (e.g., antithrombin, plasmin, urokinase, streptokinase, fibrinogen activator, thrombin), kallikrein, kinin, bradykinin antagonists, enzymes which do not act on the blood, hormones, growth factors such as bone-forming factors and cell growth factors, proteinaceous bone growth factors, clotting and anti-clotting agents, hemolysis inhibitors, and agents for treating osteoporosis.

The filler used in the invention is preferably one or more selected from among metals, ceramics, polymers, carbonaceous materials, and composites thereof. These composites are materials composed of two or more types of mutually differing substances which are strongly bonded and united by physical, chemical or mechanical mixing and joining. Illustrative examples include composite materials obtained by kneading together components made of different substances, and composite materials obtained by precipitation from a precursor solution or the like.

The filler may hold at the interior a drug or the like. Preferred examples of fillers for holding drugs include any one or more hydrogel or dried form thereof from among polyvinyl alcohol, collagen, gelatin, agar, hyaluronic acid, chitin/chitosan and polyvinyl acetate; biodegradable polymers such as polylactic acid polymers and polyethylene glycol polymers; and composites of these with calcium phosphate-based ceramics.

In the present invention, the phrase “particles held at the interior of the oriented pores” refers to particles having a particle size no larger than the diameter at the openings of the oriented pores and no smaller than the diameter at the openings of the connecting pores. The particles thus held do not necessarily have to be fixed to the walls at the interior of the porous body. The particles used in the present invention are preferably composed of one or more of the following: metals, ceramics, polymers, carbonaceous materials, and composites thereof. These composites are materials composed of two or more types of mutually differing substances which are strongly bonded and united by physical, chemical or mechanical mixing and joining. Illustrative examples include composite materials obtained by kneading together components made of different substances, and composite materials obtained by precipitation from a precursor solution or the like.

Some of the particles, or at least some portion of the particle surfaces, may be covered with at least one substance selected from among calcium phosphate, titanium oxide, alkali titanates, polymers, silane coupling agents, compounds formed by hydrolyzing a metal alkoxide, mesoporous materials, drugs, and compounds containing one or more element from among calcium, magnesium, sodium, potassium, lithium, zinc, tin, tantalum, zirconium, silicon, niobium, aluminum, iron, phosphorus and carbon. Moreover, a drug may be held in some of the particles. In the practice of the invention, the silane coupling agent may have a fluorocarbon chain or a long-chain alkyl chain, and may have a carboxyl group, an alcohol group or an amino group at the end of the chain.

Next, the group of oriented pores and the group of connecting pores in the invention are defined, and the functions that can be achieved with these are described. In the present invention, the phrase “group of oriented pores having an orientation” refers to a collection of pores, each having a lengthwise orientation that is substantially uniformly aligned in a specific direction, which pores allow infiltration by living tissue and the introduction of cells, have a structure for eliciting the necessary bodily functions at the site of implantation, are moreover able to achieve a preferred structure for regenerating the ordered structure (oriented structure) of living tissue at the interior of the porous body, and the length of which pores is greater than unity relative to the diameter at the openings of the primary pores formed at the interior of the porous body.

In the invention, the phrase “connecting pores that link together oriented pores” refers to pores which link between the ends of the oriented pores, and pores which link between the oriented pores with pores of smaller diameter than the oriented pores, enabling the passage of bodily fluids and gas bubbles. Moreover, the presence of such connecting pores, by making it possible to control the connecting structure between the pores and the wall structure, enables a structure to be constructed which is functionally compatible with the surrounding bone tissue and has the required strength.

In the present invention, “lost wax” refers to a process that uses a pattern (e.g., a tree or cluster), such as investment molding (lost-wax process) or flow molding (lost mold process). The materials for this purpose are not subject to any particular limitation. For example, the pattern may be made of dental wax or casting wax, or of a polymer such as an epoxy resin or polyurethane.

The method of manufacturing the porous bio-implant material according to the invention is described. Preferred examples of the method of manufacturing the porous bio-implant material of the invention include a method that involves stacking thin sheets of titanium, heating the stacked sheets in a vacuum at from 500 to 1500° C. for a period of from 1 to 500 minutes while applying a pressure of from 10 to 500 kg/cm², then diffusion bonding at 800° C.; and a method that involves heating a sheet of polylactic acid in open air at from 80 to 200° C. for a period of from 1 to 500 minutes while applying a pressure of from 0.1 to 10 kg/cm².

Other preferred examples of the above method of manufacture include a process in which a mold formed of dental wax is used to cast molten titanium or tantalum metal by investment molding or flow molding, thereby obtaining a metal shaped body; and a method in which a ceramic slurry or a sol-gel process precursor is cast in a porous shaped body made of a polymer such as urethane, then fired at from 300 to 1650° C. to form a ceramic shaped body. The inventive method of manufacture is not limited to these processes. For example, the above-described mold material, temperature and pressure may be suitably varied in accordance with the intended product.

In a biomaterial such as artificial bone, it is important to contribute to the formation of ordered living tissue, and also to form a porous artificial bone which will have a suitable compatibility with the mechanical properties at the site of implantation. To this end, it is important, within the porous material, both to control the direction of the pores so that they are oriented in the desired direction and also to form, in such a way as to link together the oriented pores, connecting pores which enable the passage of bodily fluids and gas bubbles. However, among prior-art biomaterials such as artificial bone, no examples whatsoever have been reported of porous biomaterials which have a three-dimensional structure wherein the spatial configuration of such oriented pores and connecting pores is controlled, and which are advantageous for infiltration by living tissue and the introduction of cells.

The biomaterial of the present invention, which satisfies the above conditions, is a material which forms a porous structure and includes as essential features: (1) a group of oriented pores in at least 50% of which a long axis direction is oriented in the same direction, to which oriented pores the infiltration by living tissue and the introduction of cells is possible, (2) connecting pores formed so as to link together the oriented pores and allow the passage therethrough of bodily fluids and gas bubbles, and (3) wherein the oriented pores are spatially configured so as not to be directly connected to other oriented pores and the connecting pores which link together the oriented pores are spatially arrayed so as not to be directly connected to other connecting pores. These features enable the construction of a porous structure suitable for the formation of hard tissue and soft tissue and for the passage of bodily fluids, etc., which porous structure, by not impeding the passage of bodily fluids and gas bubbles, promotes the supply of nutrients and oxygen. As a result, sufficient cell, tissue and vascular infiltration occurs, promoting tissue regeneration.

The porous biomaterial of the invention has a porous structure formed of oriented pores and connecting pores that link together the oriented pores. Moreover, it is critical that in at least 50% of the oriented pores the long axis direction is oriented in the same direction, and that the oriented pores are spatially configured so as not to be directly connected to other oriented pores and the connecting pores which link together the oriented pores are spatially arrayed so as not to be directly connected to other connecting pores. In this way, the oriented pores enable the infiltration by living tissue and the introduction of cells, and the connecting pores enable the passage of bodily fluids and gas bubbles, so that the group of oriented pores function as spaces for tissue, cell and vascular infiltration and the connecting pores function as spaces for the supply of nutrients and oxygen.

In the present invention, the oriented pores are spatially configured so as not to be directly connected to other oriented pores and the connecting pores which link together the oriented pores are spatially arrayed so as not to be directly connected to other connecting pores. The reason has to do with bone histology. That is, because the tissue that is regenerated forms along the direction of the oriented pores, in a spatial configuration where the oriented pores are directly connected and oriented pores having different directions of orientation mutually intersect, this would be unsuitable for tissue regeneration in which, for example, the cortical bone units of the femoral diaphysis are arrayed unidirectionally.

In the present invention, it is preferable for the long axis direction of the group of oriented pores to be facing in the same direction. Here, “to be facing in the same direction,” means that the group of oriented pores have the same degree of orientation as is observed in living tissue such as, for example, an array of bone units within the cortical bone of the femoral diaphysis. In this case, it is desirable from the standpoint of ease of design, ease of manufacture and cost that the present invention, rather than directly mimicking the orientation of bone tissue, derive the stress dispersion and the tissue direction of orientation using bone tissue as a model, and further simplify this orientation.

The greatest feature of the present invention is that, by constructing a porous body in which the orientation has been controlled in the above-described simplified manner, it is possible to create a novel biomaterial capable of satisfying all requirements: suitable for the regeneration of bone tissue, ease of biomaterial design and manufacture, and reasonable cost. With regard to orientation, although error does arise both in manufacturing and in use, such error is included within the allowable range of what is regarded in the present invention as being oriented in the same direction. However, it is desirable for any shifts in orientation on account of manufacturing error to fall within the range in the degree of orientation seen in living tissue.

In the present invention, by using a porous body having oriented pores at least 50% of which have an orientation, the infiltration by bone tissue is promoted, enabling suitable control of the morphology of the biomaterial capable of being formed there. Also, in the present invention, employing a porous structure having the above-described specific structure makes it possible to calculate, design, regulate and produce to a high precision characteristics such as the shape, structure and size of the oriented pores and connecting pores, the type of material, the abundance of pores, connecting structures and the degree of orientation. However, in cases where the oriented pores are directly connected to other oriented pores or the connecting pores which link together the oriented pores are directly connected to other connecting pores, such highly precise regulation, and the like is not possible.

The present invention, by carrying out such highly precise regulation, etc., makes it possible to construct and furnish a porous structure that supplies nutrients and oxygen and is able to suitably control tissue regeneration and the formation of hard tissue and soft tissue. These are only achievable when conditions such as the following are satisfied: the porous structure has a certain, highly ordered, spatial configuration composed of oriented pores having a high degree of orientation and connecting pores which link together the oriented pores, the spatial configuration can be suitably designed with a desired morphology and the resulting spatial morphology quantitatively controlled, and design changes therein can be freely and easily carried out.

The present invention provides the following advantages.
(1) A porous biomaterial can be obtained in which communicating pores have been formed and in which the orientation, size and shape of the pores at the interior of the porous body have been directly controlled.
(2) As a result, the passage of bodily fluids and gas bubbles is facilitated by the pores that have been formed, and a suitable scaffolding for bone tissue and vascular infiltration can be provided.
(3) This in turn enables the morphology of the living tissue that forms there to be controlled by the geometric shape of the formed pores.
(4) Through control of the geometric shape—such as the shape and orientation—of the pores and control of the pore distribution, anisotropy arises in the mechanical characteristics (strength and modulus) of the porous body, making it possible to achieve the necessary stress dispersion at the site of implantation.
(5) Through control of the geometric shape—such as the shape and orientation—of the pores and control of the pore distribution, anisotropy arises in the propagation of sound waves, vibrations and electromagnetic waves by the porous body, making it possible to achieve the necessary propagation of vibrations and electromagnetic waves at the site of implantation.
(6) Through control of the geometric shape—such as the shape and orientation—of the pores and control of the pore distribution, anisotropy arises in the attenuation of sound waves, vibrations and electromagnetic waves by the porous body, making it possible to achieve the necessary absorption of vibrations and electromagnetic waves at the site of implantation.

### BRIEF DESCRIPTION OF THE DRAWINGS

FIG. 1 shows schematic diagrams of the throughholes in the sheets according to Examples 1 to 5.

FIG. 2 are photographs of the porous bodies composed of oriented pores and connecting pores which link together the oriented pores according to Examples 1 to 4.

FIG. 3 is a schematic diagram of a porous body composed of oriented pores and connecting pores which link together the oriented pores according to Examples 1 to 4.

FIG. 4 is a schematic diagram of a porous body composed of oriented pores and connecting pores which link together the oriented pores according to Example 5.

FIG. 5 is a schematic diagram of a porous body composed of oriented pores and connecting pores which link together the oriented pores according to Example 6. The lengths of the oriented pores differ in portions (1) and (2).

FIG. 6 is a schematic diagram of a porous body composed of two-dimensionally oriented pores and connecting pores which link together the oriented pores according to Example 7.

FIG. 7 is a schematic diagram of a porous body composed of oriented pores and connecting pores which link together the oriented pores according to Example 8.

FIG. 8 is a schematic diagram of a porous body composed of oriented pores and connecting pores which link together the oriented pores according to Example 9.

FIG. 9 is a schematic diagram of a porous body composed of oriented pores and connecting pores which link together the oriented pores according to Example 10.

FIG. 10 is a schematic diagram of a porous body having honeycomb-like throughholes according to Comparative Example 1.

FIG. 11 is a schematic diagram of a porous body having a three-dimensional trabecular structure derived from a mechanical model of bone.

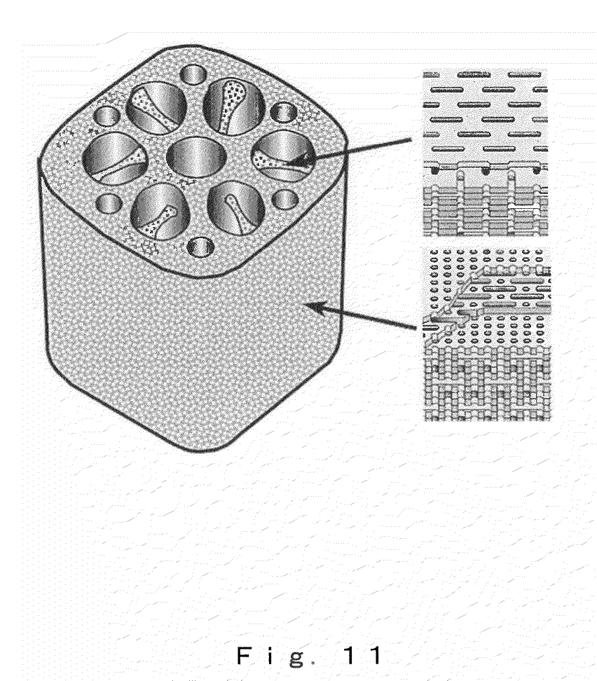

FIG. 12 shows induced tissue within the surface microspatial structure of the porous body in Example 14.

FIG. 13 shows induced tissue within the surface microspatial structure of the porous body in Example 14.

FIG. 14 shows induced tissue within the surface microspatial structure of the porous body in Example 14.

FIG. 15 shows induced tissue within the surface microspatial structure of the porous body in Example 15.

FIG. 16 is a photograph of a porous body composed of oriented pores and connecting pores which link together the oriented pores according to Example 16.

FIG. 17 is a photograph of a porous body composed of oriented pores and connecting pores which link together the oriented pores according to Example 17.

### BEST MODE FOR CARRYING OUT THE INVENTION

Next, the invention is described more fully based on examples, although the invention is not limited in any way by the following examples.

### EXAMPLE 1

### Stacking of Titanium Sheets

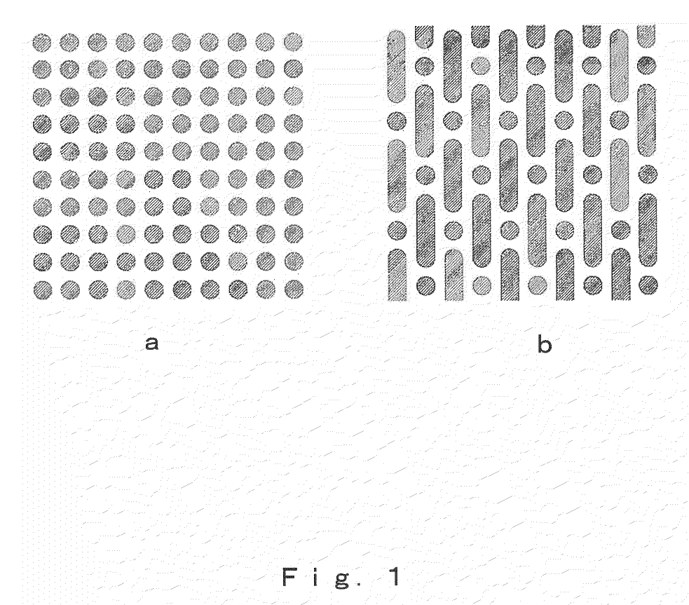

Three 100 μm thick titanium sheets having circular throughholes of 150 μm radius (shape: FIG. 1a) and three 100 μm thick titanium sheets having circular holes of 150 μm radius and throughholes of 300 μm width and 1,200 μm length (shape: FIG. 1b) were alternately stacked, and the titanium sheets were diffusion bonded to each other by heating in a vacuum at from 500 to 1,500° C. for a period of from 1 to 500 minutes while applying a pressure of from 10 to 500 kg/cm².

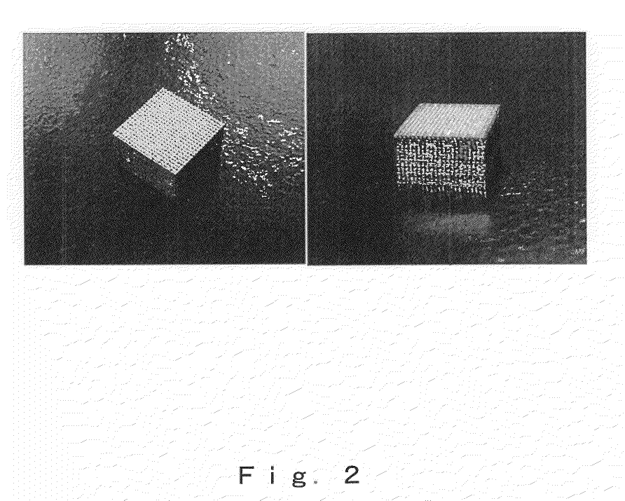

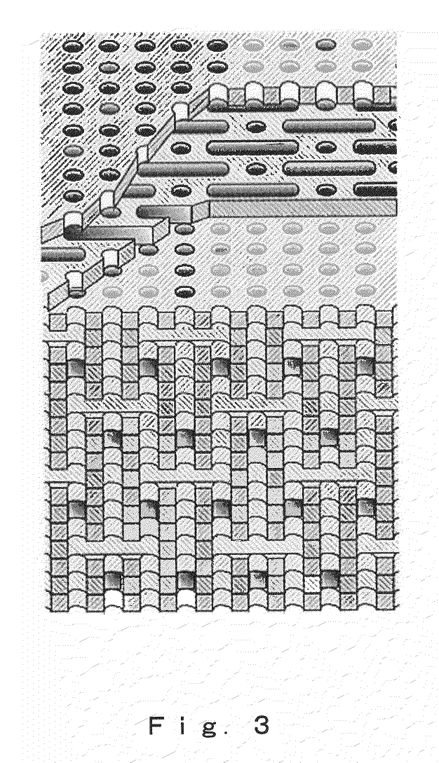

This gave a bulk porous body made of titanium characterized by being a porous body having therein a group of oriented pores of individually controlled size, shape and direction and with an orientation in one direction and also having formed therein connecting pores that link together the oriented pores and enable the passage of bodily fluids and gas bubbles, and by being formed with a controlled spatial configuration of the oriented pores and connecting pores (FIGS. 2 and 3). It was possible to control the size of the bulk porous body by means of the size of the titanium sheets that are stacked and the number of stacked layers. The porous body had a bulk density of 1.47 g/cm³ and a relative density of about 32%.

### EXAMPLE 2

### Stacking of Polylactic Acid Sheets

A 300 μm thick polylactic acid sheet having circular throughholes of 150 μm radius (shape: FIG. 1a) and a 300 μm thick polylactic acid sheet having circular holes of 150 μm radius and throughholes of 300 μm width and 1,200 μm length (shape: FIG. 1b) were stacked, and the polylactic acid sheets were fusion bonded to each other by heating in the open air at from 80 to 200° C. for a period of from 1 to 500 minutes while applying a pressure of from 0.1 to 10 kg/cm².

This gave a bulk porous body made of polylactic acid characterized by being a porous body having therein a group of oriented pores of individually controlled size, shape and direction and with an orientation in one direction and also having formed therein connecting pores that link together the oriented pores and enable the passage of bodily fluids and gas bubbles, and by being formed with a controlled spatial configuration of the oriented pores and connecting pores. It was possible to control the size of the bulk porous body by means of the size of the polylactic acid sheets that are stacked and the number of stacked layers. The porous body had a bulk density of 0.41 g/cm³ and a relative density of about 32%.

### EXAMPLE 3

### Stacking of Polylactic Acid Sheet and Titanium Sheet

A 100 μm thick titanium sheet having circular throughholes of 150 μm radius (shape: FIG. 1a) and a 300 μm thick polylactic acid sheet having circular holes of 150 μm radius and throughholes of 300 μm width and 1,200 μm length (shape: FIG. 1b) were stacked, and the sheets were fusion bonded by heating in the open air at from 80 to 200° C. for a period of from 1 to 500 minutes while applying a pressure of from 0.1 to 10 kg/cm². This gave a bulk porous body made of polylactic acid and titanium that was characterized by being a porous body having therein a group of oriented pores of individually controlled size, shape and direction and with an orientation in one direction and also having formed therein connecting pores that link together the oriented pores and enable the passage of bodily fluids and gas bubbles, and by being formed with a controlled spatial configuration of the oriented pores and connecting pores.

### EXAMPLE 4

### Stacking of Polylactic Acid Sheets and Hydroxyapatite

A 300 μm thick polylactic acid sheet having circular throughholes of 150 μm radius and a 300 μm thick polylactic acid sheet having circular holes of 150 μm radius and throughholes of 300 μm width and 1,200 μm length were stacked with hydroxyapatite particles inserted therebetween, and the sheets were fusion bonded in such a way as a envelope the apatite particles between the sheets by heating in the open air at 150° C. for 1 hour while applying a pressure of 1 kg/cm². This gave a bulk porous body made of polylactic acid and hydroxyapatite that was characterized by being a porous body having therein a group of oriented pores of individually controlled size, shape and direction and with an orientation in one direction and also having formed therein connecting pores that link together the oriented pores and enable the passage of bodily fluids and gas bubbles, and by being formed with a controlled spatial configuration of the oriented pores and connecting pores.

### EXAMPLE 5

### Changing the Porous Body Structure by Changing the Titanium Sheet Stack

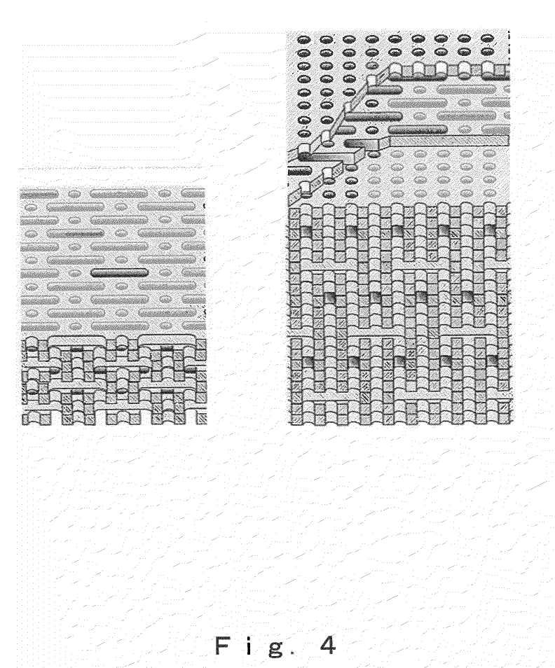

By changing the number of 100 μm thick titanium sheets having circular throughholes of 150 μm radius (shape: FIG. 1a) inserted between 100 μm thick titanium sheet having circular holes of 150 μm radius and throughholes of 300 μm width and 1,200 μm length (shape: FIG. 1b), it was possible to change the length of the oriented pores having an orientation in one direction (FIGS. 3 and 4). It was possible to control the size of the bulk body by means of the size and number of the titanium sheets stacked.

### EXAMPLE 6

### Changing the Porous Body Structure by Changing the Titanium Sheet Stack

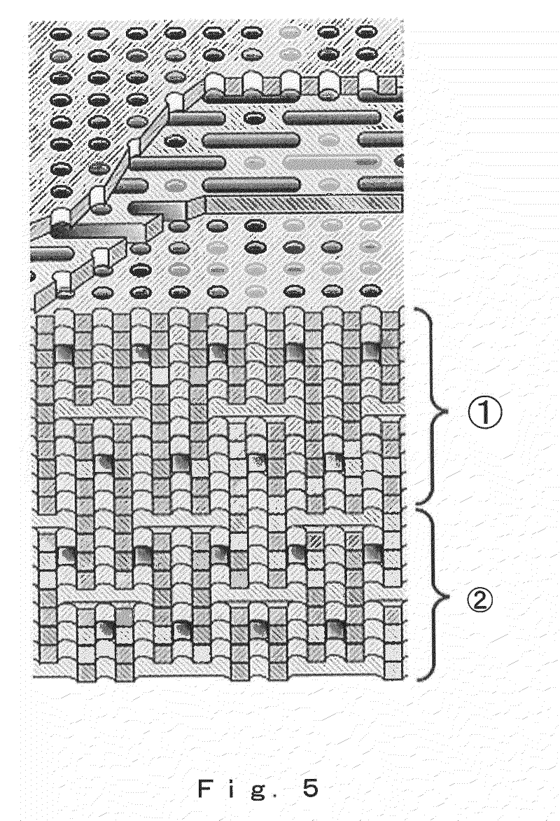

By changing the number of 100 μm thick titanium sheets having circular throughholes of 150 μm radius (shape: FIG. 1a) inserted between 100 μm thick titanium sheet having circular holes of 150 μm radius and throughholes of 300 μm width and 1,200 μm length (shape: FIG. 1b), it was possible to vary midway the length of the oriented pores having an orientation in one direction (FIG. 5). It was possible to control the size of the bulk body by means of the size and number of the titanium sheets stacked.

### EXAMPLE 7

### Stacking of Sheets Having Holes of One Shape

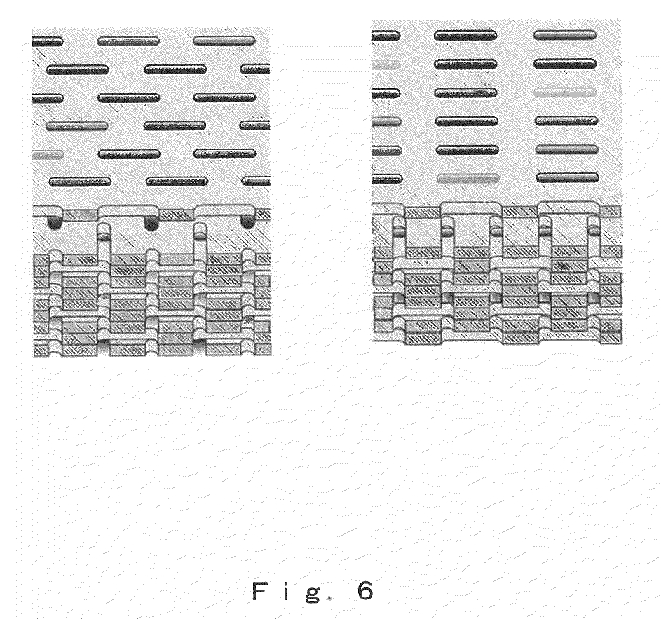

Titanium sheets having a thickness of 100 μm in which throughholes of 300 μm width and 1,200 μm length had been formed at intervals of 1,200 μm (at intervals equal to the hole length) were stacked, then diffusion bonded to each other in a vacuum at from 500 to 1,500° C. for a period of from 1 to 500 minutes while applying a pressure of from 10 to 500 kg/cm². This gave a bulk porous body made of titanium that was characterized by being a porous body having therein a group of oriented pores of individually controlled size, shape and direction and with an orientation in two directions and also having formed therein connecting pores which link together the oriented pores and enable the passage of bodily fluids and gas bubbles, and by being formed with a controlled spatial configuration of the oriented pores and connecting pores (FIG. 6).

### EXAMPLE 8

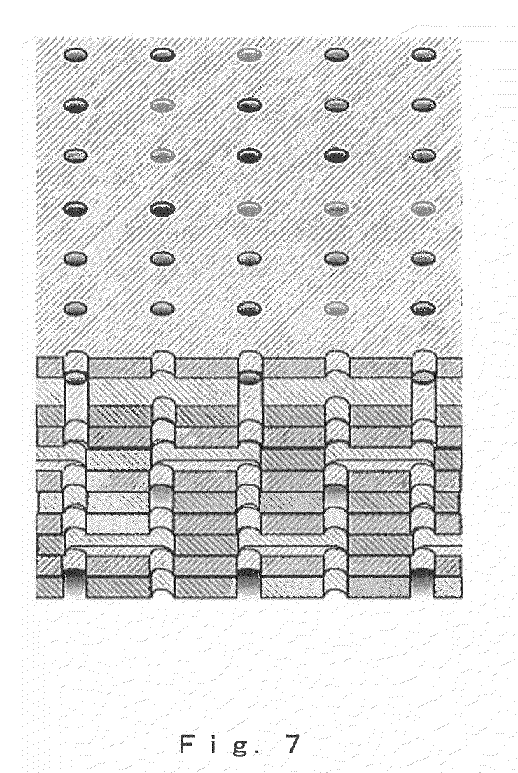

A titanium sheet in which circular throughholes of 150 μm radius are arrayed at 1,200 μm intervals and a titanium sheet in which throughholes of 300 μm width and 1,200 μm length are arrayed at 1,200 μm intervals (at intervals equal to the hole length) were stacked, then diffusion bonded to each other in a vacuum at from 500 to 1,500° C. for a period of from 1 to 500 minutes while applying a pressure of from 10 to 500 kg/cm². This gave a bulk porous body made of titanium that was characterized by being a porous body having therein a group of oriented pores of individually controlled size, shape and direction and with an orientation in three directions and also having formed therein connecting pores which link together the oriented pores and enable the passage of bodily fluids and gas bubbles, and by being formed with a controlled spatial configuration of the oriented pores and connecting pores (FIG. 7).

### EXAMPLE 9

### One-Directional Orientation, Two-Dimensional Communication

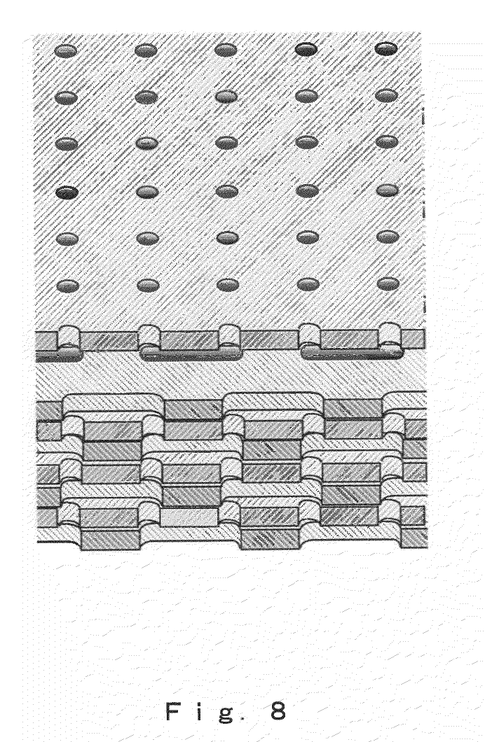

A titanium sheet in which circular throughholes of 150 μm radius are arrayed at 1,200 μm intervals and a titanium sheet in which throughholes of 300 μm width and 1,200 μm length are arrayed at 1,200 μm intervals (at intervals equal to the hole length) were stacked, then diffusion bonded to each other in a vacuum at from 500 to 1,500° C. for a period of from 1 to 500 minutes while applying a pressure of from 10 to 500 kg/cm². This gave a bulk porous body made of titanium that was characterized by being a porous body having therein a group of oriented pores of individually controlled size, shape and direction and with an orientation in one direction and also having formed therein connecting pores which link together the oriented pores and enable the passage of bodily fluids and gas bubbles, and by being formed with a controlled spatial configuration of the oriented pores and connecting pores (FIG. 8).

### EXAMPLE 10

### Change in Direction of Orientation

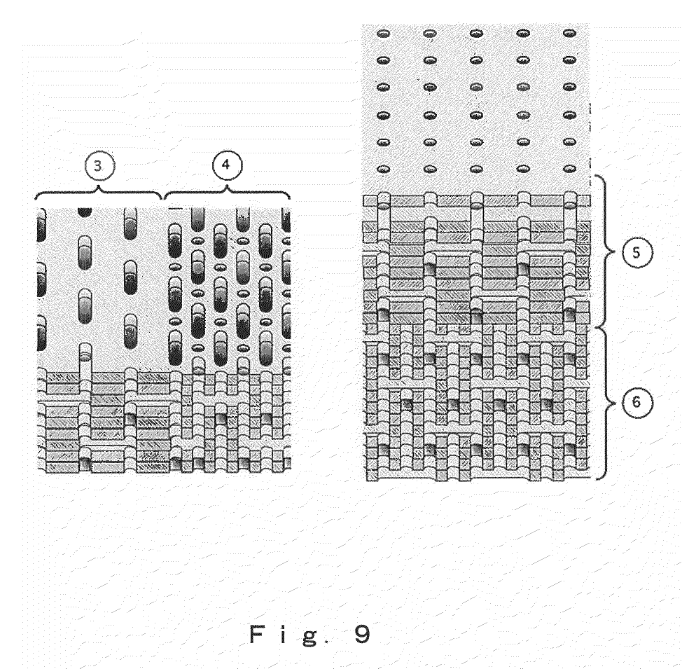

Titanium sheets having an array of throughholes in a plurality of patterns within each sheet were stacked (FIG. 9a), and three or more types of titanium sheets having differing throughhole array patterns were stacked (FIG. 9b). In each case, the stacked titanium sheets were diffusion bonded to each other in a vacuum at from 500 to 1,500° C. for a period of from 1 to 500 minutes while applying a pressure of from 10 to 500 kg/cm², thereby forming bulk porous bodies made of titanium wherein the direction of orientation by the oriented pores changes at the interior of the porous material and which had communicating pores with an orientation.

### COMPARATIVE EXAMPLE 1

### Porous Body Having Honeycomb-Type Throughholes

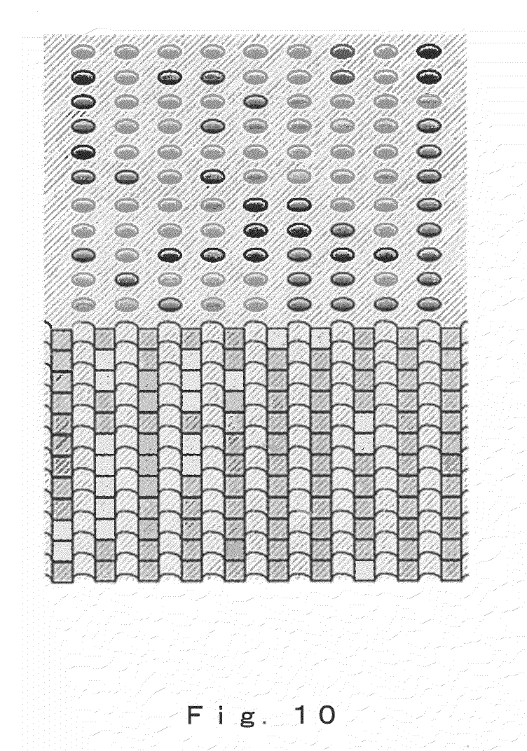

Titanium sheets with a thickness of 100 μm and having circular throughholes of 150 μm radius (shape: FIG. 1a) were stacked, then diffusion bonded to each other in a vacuum at from 500 to 1,500° C. for a period of from 1 to 500 minutes while applying a pressure of from 10 to 500 kg/cm², thereby forming a honeycomb-like bulk porous body made of titanium and having throughholes with an orientation in one direction. The individual throughholes were isolated; it was not possible to form communicating holes (FIG. 10).

### EXAMPLE 11

### Mold for Lost-Wax Process

A 500 μm thick wax sheet having 500 μm circular throughholes and a 500 μm thick wax sheet having 500 μm circular throughholes and holes with a width of 500 μm and a length of 2,000 μm were stacked, then fusion bonded at from 40 to 150° C. while applying a pressure of from 0.1 to 10 kg/cm². This gave a mold that was characterized by being a porous body having therein a group of oriented pores of individually controlled size, shape and direction and with an orientation in one direction and also having formed therein connecting pores which link together the oriented pores and enable the passage of bodily fluids and gas bubbles, and by being formed with a controlled spatial configuration of the oriented pores and connecting pores. It was possible to control the size of the bulk body by the size and number of the sheets that are stacked.

### EXAMPLE 12

### Mold for Lost-Wax Process

A 500 μm thick sheet of epoxy resin having 500 μm circular throughholes and a 500 μm thick sheet of epoxy resin having 500 μm circular throughholes and holes with a width of 500 μm and a length of 2,000 μm were stacked, then bonded. This gave a mold that was characterized by being a porous body having therein a group of oriented pores of individually controlled size, shape and direction and with an orientation in one direction and also having formed therein connecting pores which link together the oriented pores and enable the passage of bodily fluids and gas bubbles, and by being formed with a controlled spatial configuration of the oriented pores and connecting pores. It was possible to control the size of the bulk body by the size and number of the sheets that are stacked.

### EXAMPLE

### 13

### Shock Absorbing Material

A 3 mm thick sheet of expanded polystyrene having 1 mm circular throughholes (shape: FIG. 1a) and a 3 mm thick sheet of expanded polystyrene having 1 mm circular throughholes and holes of a width of 1 mm and a length of 20 mm (shape: FIG. 1b) were stacked, and the sheets of expanded polystyrene were bonded to each other with an adhesive. This gave a mold that was characterized by being a porous body having therein a group of oriented pores of individually controlled size, shape and direction and with an orientation in one direction and also having formed therein connecting pores which link together the oriented pores, and by being formed with a controlled spatial configuration of the oriented pores and connecting pores. It was possible to control the size of the bulk body by the size and number of the sheets of expanded polystyrene that are stacked.

### EXAMPLE 14

### Animal Test of Implant Having Oriented Microspatial Structure

Implants measuring 4×3×5 mm³ in which oriented microspaces having an average width of about 180 μm and a length of 1,200 μm were formed so as to be connected by microspaces having an average width of about 180 μm were implanted in a bone defect formed as a hole of 5 mm radius and 5 mm depth near the proximal end of the neckbone in healthy, 12-week-old male SPF rabbits, following which the periosteum, subcutaneous tissue and skin were sutured. Seven days, two weeks or four weeks following placement of the implants, the animals were euthanized by bleeding under sodium pentobarbital (approx. 50 mL/kg i.v.) anesthesia, and the cervical implant site was removed and fixed in 10% neutral buffer formalin. After fixing, the implant site was rendered into a half-decalcified state by the ion exchange method, following which sections having a thickness of about 3 μm were prepared. The sections were hematoxylin-eosin stained, then morphologically evaluated.

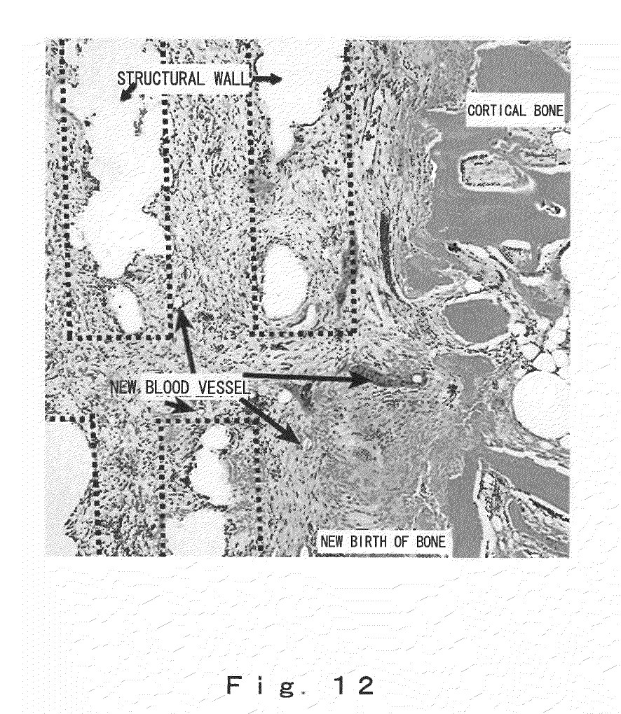

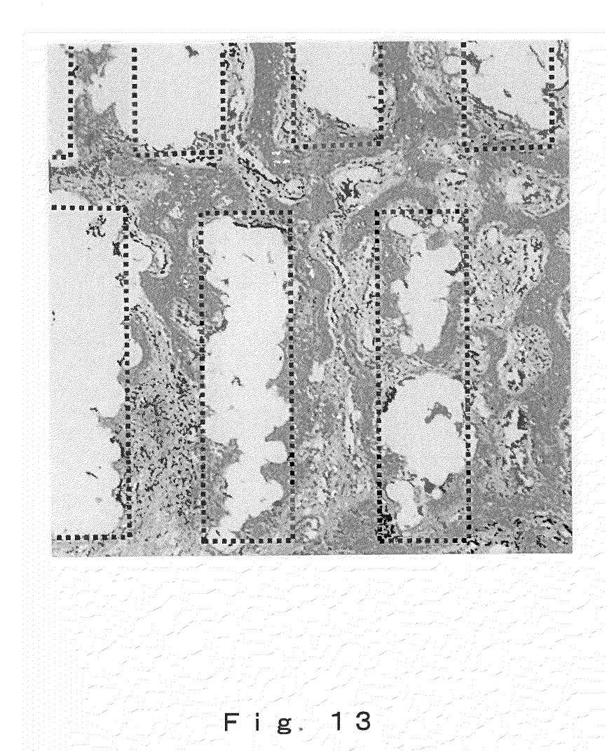

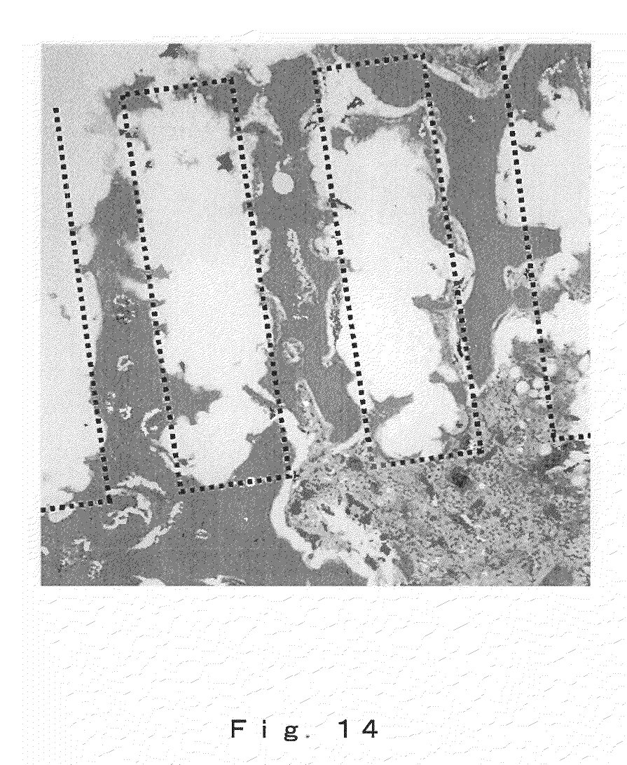

In these evaluations, on day 7 of implantation, the infiltration by granulation tissue accompanied by new blood vessels was observed within oriented microspaces at the compact bone level (FIG. 12); in week 2, the formation of new bone along the wall of the structure arose (FIG. 13); and in week 4, the formation of bone tissue accompanied by blood vessels was observed at the interior of the oriented microspaces (FIG. 14). The bone tissue that had formed was oriented tissue which formed using as a template the structure of the oriented microspaces in the implant.

### EXAMPLE 15

### Animal Test of Implant Having Isotropic Microspatial Structure

Implants measuring 4×3×5 mm³ in which microspaces having an average width of about 390 μm were formed in an isotropic arrangement and connected by microspaces having an average width of about 230 μm were implanted in a bone defect formed as a hole of 5 mm radius and 5 mm depth near the proximal end of the neckbone in health, 12-week-old male SPF rabbits, following which the periosteum, subcutaneous tissue and skin were sutured. Four weeks following placement of the implants, the animals were euthanized by bleeding under sodium pentobarbital (approx. 50 mL/kg i.v.) anesthesia, and the cervical implant site was removed and fixed in 10% neutral buffer formalin. After fixing, the implant site was rendered into a half-decalcified state by the ion exchange method, following which sections having a thickness of about 3 μm were prepared. The sections were hematoxylin-eosin stained, then morphologically evaluated.

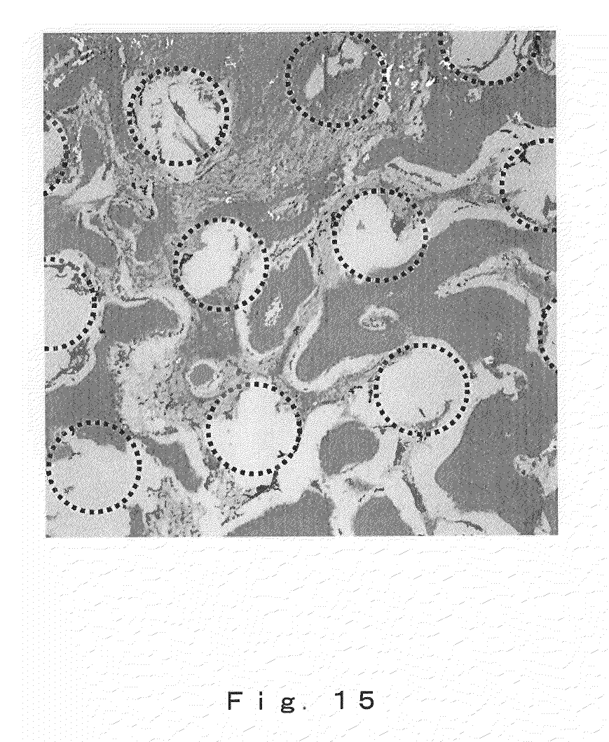

In these evaluations, the formation of bone tissue and bone marrow tissue accompanied by new blood vessels was observed within isotropically arranged spaces at the compact bone level (FIG. 15), but oriented tissue did not form in the spaces having an isotropic structure.

### EXAMPLE 16

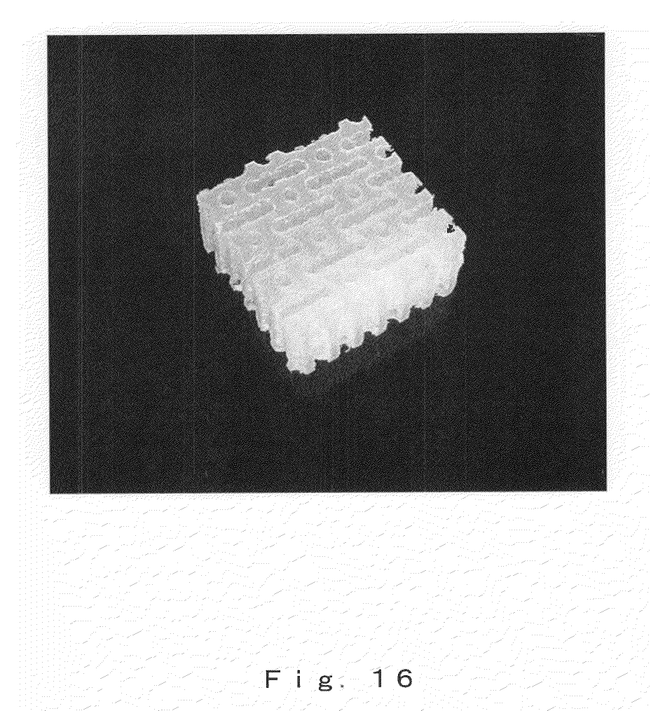

A 300 μm thick sheet of polylactic acid having circular throughholes of 500 μm radius and a 300 μm thick polylactic acid sheet having circular throughholes of 500 μm radius and holes of a width of 1,000 μm and a length of 4,000 μm were stacked, following which the sheets were fusion bonded by heating in the open air at from 80 to 150° C. for a period of from 10 to 60 minutes while applying a pressure of from 0.1 to 1 kg/cm². This gave polylactic acid that was characterized by being a porous body having therein a group of oriented pores of individually controlled size, shape and direction and with an orientation in one direction and also having formed therein connecting pores which link together the oriented pores and enable the passage of bodily fluids and gas bubbles, and by being formed with a controlled spatial configuration of the oriented pores and connecting pores (FIG. 16).

### EXAMPLE 17

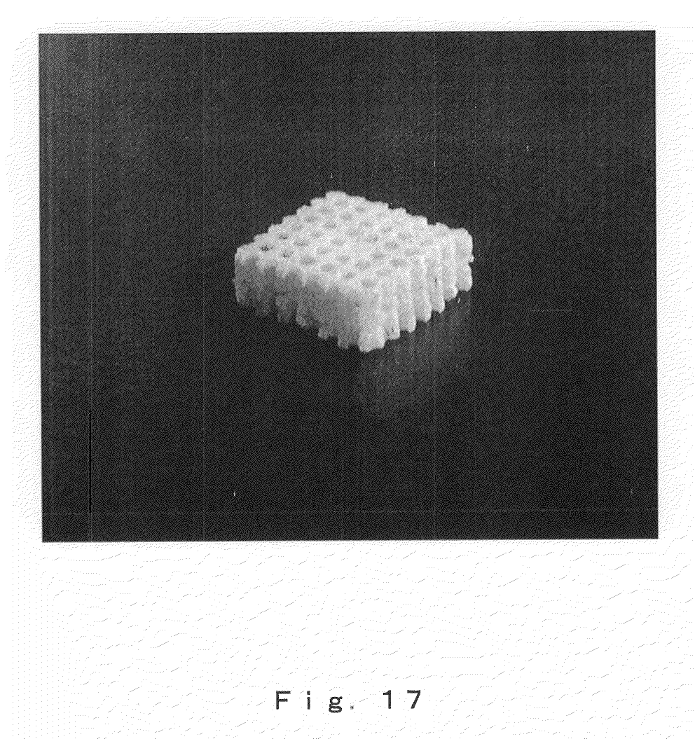

A 300 μm thick sheet composed of alumina fibers and silica fibers and having therein circular throughholes of 500 μm radius and a 300 μm thick sheet composed of alumina fibers and silica fibers and having therein circular throughholes of 500 μm radius and holes of a width of 1,000 μm and a length of 4,000 μm were stacked while being bonded with an inorganic adhesive or a cyanoacrylate adhesive so as to obtain a porous body in which the sheets have been joined together. This gave ceramic porous body or a ceramic-polymer composite porous body that was characterized by being a porous body having therein a group of oriented pores of individually controlled size, shape and direction and with an orientation in one direction and also having formed therein connecting pores which link together the oriented pores and enable the passage of bodily fluids and gas bubbles, and by being formed with a controlled spatial configuration of the oriented pores and connecting pores (FIG. 17).

### INDUSTRIAL APPLICABILITY

As described in detail above, the present invention relates to a biocompatible implant material and a method of manufacture thereof. The invention makes it possible to form a porous bio-implant material in which communicating pores of directly designed and controlled pore orientation, size and shape have been formed at the interior of a porous body. As a result, this invention is able to provide a bio-implant material which, by means of the geometric shape that is formed, can control the morphology of the living tissue to be formed there. The present invention is able to provide a bio-implant material wherein, through control of the geometric shape—including the shape and orientation of the pores—and control of the distribution of the pores, anisotropy in the mechanical characteristics (strength and modulus) of the porous body arises and stress diffusion is controlled.

1. A porous biomaterial with controlled orientation, characterized (1) by having a group of oriented pores, at least 50% of which in a long axis direction is oriented in the same direction, (2) by having connecting pores that are formed so as to link together the oriented pores and enabling the passage of bodily fluids and gas bubbles, and (3) by spatial configuration in which the oriented pores are not directly connected to other oriented pores and the connecting pores which link together the oriented pores are not directly connected to other connecting pores.

2. The biomaterial of claim 1, which is made of metal, polymer, ceramic or a composite of any two or more thereof.

3. The biomaterial of claim 1, which is formed of stacked sheets.

4. The biomaterial of claim 3, wherein each sheet to be stacked has a thickness of from 10 μm to 2 mm, or up to one-half the entire thickness of the biomaterial.

5. The biomaterial of claim 3, wherein the sheets to be stacked have a pore size with a minimum width, in a direction perpendicular to the surface of the sheets, in a range of from 0.1 μm to 1 mm.

6. The biomaterial of claim 3, wherein the sheets to be stacked have a pore size with a maximum width, in a direction perpendicular to the surface of the sheets, in a range of from 10 μm to 10 mm.

7. The biomaterial of claim 3, wherein the sheets to be stacked have a pore frequency of from 1 to 250,000 per square centimeter.

8. The biomaterial of claim 3, wherein the sheets to be stacked are made of metal, polymer, ceramic or a composite of any two or more thereof.

9. The porous biomaterial of any of claims 1 to 8, wherein at last a portion of walls of the oriented pores and/or connecting pores contains, or is covered with, at least one selected from among calcium phosphate, titanium oxide, alkali titanates, polymers, silane coupling agents, compounds formed by hydrolyzing a metal alkoxide, mesoporous materials, drugs, and compounds containing one or more element from among calcium, magnesium, sodium, potassium, lithium, zinc, tin, tantalum, zirconium, silicon, niobium, aluminum, iron, phosphorus and carbon.

10. The biomaterial of claim 9, wherein at least a portion of the walls of the oriented pores and/or connecting pores has been rendered porous by anodization.

11. The biomaterial of claim 9, wherein at least a portion at the interior of the oriented pores and the connecting pores which link together the oriented pores holds at least one type of filler composed of one or more selected from metal, ceramic, polymer or a composite thereof.

12. The biomaterial of claim 9, wherein at least a portion at the interior of the oriented pores and the connecting pores which link together the oriented pores holds at least one type of particle composed of one or more selected from metal, ceramic, polymer or a composite thereof.

13. A process for manufacturing the porous biomaterial of any of claims 1 to 8, comprising: using as a mold a shaped body, which has a structure obtained by stacking and joining together sheets containing pores of at least two types of shape, array pattern and frequency, the pores being of differing width-to-length ratios, while controlling pore positions in the sheets, and which has, at the interior of a porous body, a group of oriented pores of individually controlled size, shape and direction, and is formed therein with connecting pores linking together the oriented pores, and is formed with a spatial configuration in which the oriented pores are not directly connected to other oriented pores and the connecting pores which link together the oriented pores are not directly connected to other connecting pores;
filling the pores with a slurry of metal, ceramic, polymer or a composite thereof; then removing the shaped body serving as the mold by sintering or by dissolution with a solvent so as to give a biomaterial which has, at the interior of a porous body, a group of oriented pores of individually controlled size, shape and direction, is formed therein with connecting pores linking together the oriented pores, and is formed with a spatial configuration in which the oriented pores are not directly connected to other oriented pores and the connecting pores which link together the oriented pores are not directly connected to other connecting pores.

14. A process for manufacturing a porous biomaterial, comprising: casting a metal or a ceramic particle-containing metal by using, as a lost wax mold, a shaped body, which has a structure obtained by stacking and joining together sheets containing pores of at least two types of shape, array pattern and frequency, the pores being of differing width-to-length ratios, while controlling pore positions in the sheets, and which has, at the interior of a porous body, a group of oriented pores of individually controlled size, shape and direction, is formed therein with connecting pores linking together the oriented pores, and is formed with a spatial configuration in which the oriented pores are not directly connected to other oriented pores and the connecting pores which link together the oriented pores are not directly connected to other connecting pores, so as to produce a biomaterial which has, at the interior of a porous body, a group of oriented pores of individually controlled size, shape and direction, has formed therein connecting pores linking together the oriented pores, and is formed with a spatial configuration in which the oriented pores are not directly connected to other oriented pores and the connecting pores which link together the oriented pores are not directly connected to other connecting pores.

15. A bio-implant which at least partially comprises the biomaterial of any one of claims 1 to 12.

16. The bio-implant of claim 15, which has a three-dimensional trabecular structure derived from a mechanical model of a bone.

17. A cell medium support which at least partially comprises the biomaterial of any one of claims 1 to 12.

18. A mold for the porous biomaterial of claims 1 to 12, the mold comprising a shaped body, which has a structure obtained by stacking and joining together sheets containing pores of at least two types of shape, array pattern and frequency, the pores being of differing width-to-length ratios, while controlling pore positions in the sheets, and which has, at the interior of a porous body, a group of oriented pores of individually controlled size, shape and direction, is formed therein with connecting pores linking together the oriented pores, and is formed with a spatially configuration in which the oriented pores are not directly connected to other oriented pores and the connecting pores which link together the oriented pores are not directly connected to other connecting pores.

19. The biomaterial of claim 1, which is formed therein with holes other than the oriented pores and the connecting pores formed so as to link together the oriented pores.

20. The biomaterial of claim 1, which is of a size where the minimum length of the oriented pores in any cross-section is from 1 to 1,000 μm.

21. The biomaterial of claim 1, wherein the porous biomaterial is a shock absorbing material which has been controlled to a size where the minimum length of the oriented pores in any cross-section is from 1 to 30 mm.

22. The biomaterial of claim 1, which is made of titanium or a titanium alloy.

23. The biomaterial of claim 1, which is made of calcium phosphate.
